(19) United States
(12) Patent Application Publication
(10) Pub. No.: US 2009/0030335 A1
(43) Pub. Date: Jan. 29, 2009

(54) METHOD AND APPARATUS FOR RESPIRATORY MONITORING

(75) Inventor: Gert KUCHLER, Randersacker (DE)

Correspondence Address: BROWDY AND NEIMARK, P.L.L.C.;624 NINTH STREET, NW, SUITE 300, WASHINGTON, DC, 20001-5303, US

(73) Assignee: SOMNOmedics GmbH, Randersacker (DE)

(21) Appl. No.: 12/181,293

(22) Filed: Jul. 28, 2008

(30) Foreign Application Priority Data
Jul. 28, 2007 (DE) 10 2007 035 549.3

(51) Int. Cl. A61B 5/08; A61B 19/00; A61M 16/00

(52) U.S. Cl. 600/534; 128/898; 128/204.22; 600/529

(57) ABSTRACT

The method and the apparatus are used for monitoring a patient's breathing. A respiratory movement of the patient is detected by means of a thoracic expansion sensor that is placed near the rib cage and by means of an abdominal expansion sensor that is placed near the abdomen. Moreover, a phase difference is detected between a thoracic measuring signal delivered by the thoracic expansion sensor and an abdominal measuring signal delivered by the abdominal expansion sensor. The phase difference thus detected is monitored to determine whether it exceeds at least a phase threshold or whether it increases over time, and the presence of a disorder in the respiratory tract of the monitored patient is recognized on the basis of an increasing phase difference.

### BACKGROUND OF THE INVENTION

1. Field of the Invention

The invention concerns a method and a device for patient monitoring. This method enables a respiratory movement of the patient to be detected by means of a thoracic expansion sensor that is placed near the rib cage and an abdominal expansion sensor that is placed near the abdomen. In order to detect a respiratory movement of the patient, the apparatus comprises a thoracic expansion sensor to be placed near the rib cage and an abdominal expansion sensor to be placed near the abdomen as well as an evaluation and control unit to which the thoracic expansion sensor and the abdominal expansion sensor are connected.

2. Background Art

A method and an apparatus of this type are in particular used for diagnosing respiratory sleep disorders or for determining obstructions by means of Ear, Nose and Throat diagnostics. Obstructions or partial obstructions, respectively, of the upper airways, i.e. near the trachea or in the area of the larynx, may result in substantial levels of sleep disturbance and sleep fragmentation which greatly impair the process of sleep. The restorative stages of deep sleep become shorter or do not occur at all. Therefore, a comprehensive detection of such respiratory disorders is an important goal of sleep diagnostics and therapy.

However, in particular partial obstructions are virtually unrecognizable from an amplitude reduction (=hypopnea) of the detected respiratory flow. The conventional method of analyzing the flow measuring signal only provides a limited amount of information with respect to the level of obstruction. Furthermore, although a complete apnea may be recognized from the vanishing flow measuring signal, this method is however quite unable to provide reliable information as to the cause thereof. In particular, it is impossible for partial obstructions to be detected from the flow measuring signal if a decrease in respiratory flow is compensated for by an increased respiratory effort of the patient. Other conventional methods of determining the level of obstruction are based on an analysis of the snoring activities. The correlation between snoring and the level of obstruction is however quite small, with the consequence that the results produced by these analytical methods are often unsatisfactory.

### SUMMARY OF THE INVENTION

Thus it is an object of the invention to disclose a method of the above type which enables a more precise detection of a respiratory disorder to be achieved.

In order to achieve this object, a method is disclosed for monitoring a patient. The inventive method comprises detection of a phase difference between a thoracic measuring signal delivered by the thoracic expansion sensor and an abdominal measuring signal delivered by the abdominal expansion sensor. Moreover, the detected phase difference is checked to determine whether it exceeds at least a phase threshold or whether it increases over time. A phase difference that exceeds the phase threshold or an increase in phase difference is indicative of a respiratory disorder of the monitored patient.

The invention is based on the fact that the phase difference of the two expansion measuring signals enables very precise statements to be made about the presence and in particular the severity of the respiratory disorder. The respiratory disorder, which is very easily detectable by means of this method, may for example be an obstruction in particular of the upper airways such as the trachea which, depending on the severity thereof, may cause an obstructive hypopnea or apnea. When the airways are unobstructed, the thoracic and the abdominal measuring signal are virtually in phase. It was discovered according to the invention that an obstruction of the upper airways results in a phase difference between these two measuring signals which is so significant that it may be detected and evaluated. Moreover, this phase difference in particular also depends on the severity of the respiratory disorder. The phase difference increases with the level of obstruction and reaches a value of almost 180° when the airways are completely obstructed. For instance, a phase difference that exceeds the phase threshold, which may for example amount to between 50° and 70°, preferably to approximately 60° depending on the sensors used, is clearly indicative of a respiratory disorder, in particular a partial obstruction. In addition or as an alternative thereto, increases in phase difference, in particular fluctuative or recurrent increases thereof, may be detected and evaluated. According to the invention, such a noticeable behavior of the phase difference is detected and recorded if necessary which requires a comparatively low amount of effort. A phase comparison of two measuring signals is easily performed by simple means. Therefore the inventive method is also suitable for automated patient monitoring, for example. In addition to that, the method may advantageously be used in combination with a sleep study and/or therapy.

In a favorable embodiment, the phase difference is monitored continuously; in particular, the phase difference is also monitored to determine whether it changes over time. Monitoring the phase difference, in particular when several phase thresholds are set, allows a distinction to be made between chronic and (partial) obstructions that only occur temporarily. A chronic partial obstruction may result in a continuous phase difference amounting to a value other than zero between the thoracic and the abdominal measuring signal. A permanent phase difference caused by such a chronic respiratory disorder may in particular be recorded as basic phase difference when monitoring starts, which is then stored in the control and evaluation unit. This basic phase difference may for example amount to approximately 30°. Accordingly, the phase threshold that serves to detect another obstruction in addition to the chronic partial obstruction is preferably set to a higher level than the basic phase difference. By means of the varying phase difference, the occurrence of an (additional) obstruction can be recognized very easily. In particular, a continuous monitoring of the phase difference gradient enables imminent obstructions to be detected at an early stage. In particular in connection with a CPAP ventilator, this allows counter measures to be taken so as to prevent imminent obstructions, thus ensuring that the patient's sleep is not affected at all or at least to a lower extent.

According to another preferred embodiment, the phase difference is continuously monitored to determine whether a time interval of the phase difference comprises a section of in particular continuously increasing phase difference that is immediately followed by a section of in particular rapidly decreasing phase difference. Such a signal curve of phase difference over time indicates an obstruction of a patient's airways that initially increases when the patient is asleep for example. If the level of obstruction becomes too severe, the patient wakes up to make systematic and conscious respiratory efforts in order to overcome the (partial) obstruction, causing an abrupt decrease in the detected phase difference. Such a sleeping behavior, which is easily detectable by the typical signal curve described above, is less restorative as a result of the recurrent arousal reactions and should therefore also be recorded in the sleep study.

Furthermore, it is also conceivable to record a period of time in which the phase difference exceeds the phase threshold. This enables short-time and therefore irrelevant respiratory disorders to be ruled out. In particular, at least one threshold may be set in relation to the period of time; a disorder is classified as significant if this threshold is exceeded. For example, a minimum threshold is set to 10 seconds and a maximum threshold is set to 120 seconds. If the detected phase difference exceeds the phase threshold for more than the above minimum threshold but for less than the above maximum threshold, the disorder is regarded as a respiratory obstructive event and is therefore preferably automatically included in a respiratory report that is established by the control and evaluation device. Strong short-term variations in the time signal of the phase difference may furthermore advantageously be neglected. To this end, the detected time signal of the phase difference is in particular subject to a low-pass filtering or a smoothing operation. For example, a moving average operation is used. Such a smoothing operation contributes to an increase in measurement reliability and helps to avoid diagnostic errors.

According to another preferred embodiment, the current degree of severity of the disorder is determined on the basis of an in particular current value of phase difference. This enables a reliable and comprehensive diagnosis of the respiratory disorder, in particular of an obstruction, to be established in a simple manner without impairment of the patient. Advantageously, this may take place automatically in the control and evaluation unit, i.e. in particular without any medical staff involved. Between the level of obstruction and the phase difference, there is in particular a unique relationship which may advantageously also be stored in the control and evaluation unit.

Advantageously, a position of the patient's body is recorded as well. It was discovered that the position is a parameter which influences the occurrence of obstructive respiratory disorders, with obstructions occurring more often when in the supine position. Recording the position is therefore advantageous in terms of a comprehensive monitoring of obstructions. These additional measuring values may be used as an additional source of information.

In another preferred embodiment, amplitudes of the thoracic measuring signal and the abdominal measuring signal may also be monitored to determine whether they fall below an amplitude threshold. This examination in particular focuses on whether this amplitude condition is fulfilled by both measuring signals. Amplitudes falling below the amplitude threshold are indicative of a dysfunctional respiratory control of the monitored patient; the dysfunction is detected and, if necessary, recorded in the control and evaluation unit. Such events are probably not so much caused by obstructed airways but by a complete failure of the respiratory muscles. This disorder presents itself in the form of a central apnea. In analogy to an obstructive apnea, the flow measuring signal of the respiratory flow, which is advantageously monitored as well, shows a vanishing or at least only very low amplitude in the event of a central apnea.

Furthermore, an alternative embodiment is favorable in which a respiratory flow of the patient is recorded and taken into account when assessing the disorder. As described above, the flow measuring signal thus detected allows determination of whether the respiratory activity, and thus the oxygen supply, are merely reduced (=hypopnea) or completely disrupted (=apnea). The additionally recorded respiratory flow may thus advantageously be taken into account when assessing the severity of an obstruction that is detected on the basis of the phase difference. In particular a partial obstruction may thus be very easily be distinguished from a complete obstruction.

According to another preferred embodiment, a counter measure, in particular an increase of a ventilation pressure, is executed in particular automatically when a disorder is recognized in the respiratory tract. In particular if the patient is connected to a ventilation apparatus and this apparatus is also controlled by the control and evaluation unit, this enables measures to be taken very quickly in the event of an imminent obstruction by increasing a ventilation pressure, for example. This may in particular take place in steps, preferably in steps of 1 mbar, thus preventing the obstruction from getting worse, and consequently the patient from waking up. Above all, this avoids disturbance of the restful stages of deep sleep which are particularly important.

Another object of the invention is to provide an apparatus of the above type which enables a more precise detection of a respiratory disorder to be carried out.

In order for this object to be achieved, an apparatus is provided for monitoring a patient, the apparatus comprising a thoracic expansion sensor to be placed near the rib cage and an abdominal measuring sensor to be placed near the abdomen, which are each used to detect a respiratory movement of the patient, as well as an evaluation and control unit to which the thoracic expansion sensor and the abdominal measuring sensor are connected. The evaluation and control unit is designed such as to determine a phase difference between a thoracic measuring signal delivered by the thoracic expansion sensor and an abdominal measuring signal delivered by the abdominal expansion sensor, to check whether the determined phase difference exceeds at least a phase threshold or increases over time, and to recognize a respiratory disorder in the monitored patient on the basis of a phase difference that exceeds the phase threshold or on the basis of an increase in phase difference.

Other features, advantages and details of the invention are set out in the ensuing description of embodiments by means of the drawings.

### BRIEF DESCRIPTION OF THE DRAWINGS

FIG. 1 shows an embodiment of an apparatus for monitoring a patient by using two elastic belts for detecting the respiratory movements;

FIG. 2 shows a simplified model of the unobstructed respiratory tract;

FIG. 3 the model of FIG. 2 when the upper airways are obstructed;

FIGS. 4 to 6 timing diagrams of measuring signals of the elastic belts according to FIG. 1 and a flow sensor that is additionally provided for detecting the respiratory flow;

FIG. 7 shows a relationship between a phase difference between the measuring signals provided by the elastic belts according to FIG. 1 and an opening surface of the airways; and

FIG. 8 shows a timing diagram of a phase difference between the measuring signals provided by the elastic belts according to FIG. 1 with increasing sections that are followed by abruptly decreasing sections.

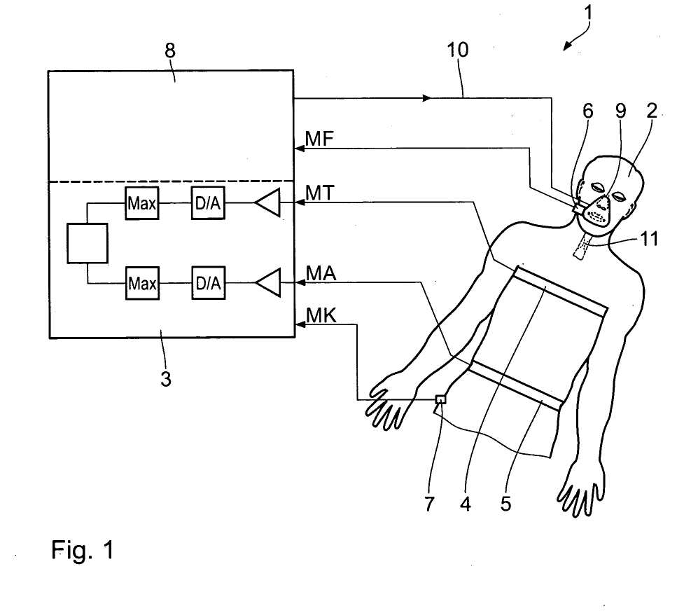

Equal components in FIGS. 1 to 8 are designated by the same reference numerals.

### DESCRIPTION OF THE PREFERRED EMBODIMENTS

FIG. 1 shows an embodiment of an apparatus 1 for monitoring a patient 2. The apparatus 1 in particular serves for monitoring the sleep of the patient 2. The apparatus 1 comprises a central control and evaluation device 3 to which two expansion sensors in the shape of a thoracic-expansion measuring belt 4 and an abdominal-expansion measuring belt 5 are connected. The thoracic-expansion measuring belt 4 is placed near the patient's 2 rib cage and the abdominal-expansion measuring belt is placed near the patient's 2 abdomen. Both measuring belts 4 and 5 detect the patient's 2 respiratory movements.

In the embodiment, both the thoracic- and the abdominal-expansion measuring belt 4 or 5, respectively, are designed as strain gauges. Alternative embodiments are however also possible. In this respect, inductive pressure sensors as they are commonly used in terms of respiratory measurement by means of inductive plethysmography are applicable. Likewise, these expansion measurements may also be carried out using optical sensors.

Preferably, the sensors used for detection of the thoracic and abdominal movements, i.e. the expansion measuring belts 4 or 5, respectively, should deliver a thoracic measuring signal MT or an abdominal measuring signal MA, respectively, wherein the phasing thereof is independent of the respiratory frequency and shall be as constant as possible as long as constant measuring conditions are maintained during the monitoring period. In particular, there should be no changes in phase behavior caused by ageing or a contamination of the sensor element or the connection cable.

The apparatus 1 further comprises a flow sensor 6 that detects the respiratory flow as well as a position sensor 7 that detects a position of the patient's 2 body. The flow sensor 6 and the position sensor 7 are optional components which are also connectable to the evaluation and control unit 3. The flow sensor 6 delivers a flow measuring signal MF to the evaluation and control unit 3 that serves as a measure for the detected respiratory flow. The position sensor 7 delivers a position measuring signal MK to the evaluation and control unit 3.

Moreover, the apparatus 1 may also be designed as a ventilation apparatus or comprise a subunit 8 that supports the patient's 2 breathing. Possible alternatives of the ventilation apparatus include the CPAP and the BiPAP type, wherein the subunit 8 used for respiratory support may either be a separate apparatus or—as shown in FIG. 1—a component of the central control and evaluation unit 3. The flow sensor 6 is in particular part of the subunit 8 for respiratory support. The latter further comprises a respiratory mask 9 and/or at least a hose 10 for the supply of additional air or oxygen.

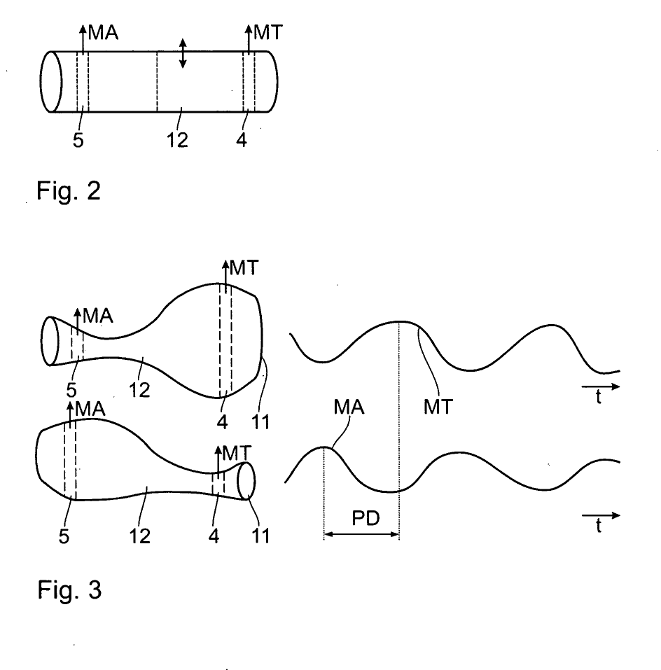

The following is a more detailed description of the function and particular advantages of the apparatus 1 by means of FIGS. 2 to 7.

The apparatus 1 detects, assesses and records respiratory disorders which may occur during the sleep. If required, the apparatus 1 may also have a controlling effect in order to eliminate a recognized respiratory disorder by increasing a ventilation pressure. All this occurs in an automated manner, i.e. without any medical staff involved. The apparatus 1 is in particular designed such as to recognize respiratory disorders that present themselves as (partial) obstructions of the upper airways.

Such an obstruction 11 may for example occur in the trachea. Depending on the severity thereof, the obstruction may result in a reduced respiratory flow (=partial obstruction with hypopnea) or a respiratory arrest (=complete obstruction with apnea). It is in particular impossible to recognize a partial obstruction by simply evaluating the flow measuring signal MF. Despite that, such a partial obstruction may also result in a disrupted sleep and may cause the patient 2 to wake up.

The schematic representations of FIGS. 2 and 3 illustrate an unobstructed respiratory tract as well as an occurring obstruction 11. In these simplified representations, the respiratory tract 12 is shown as a cylindrical hose comprising a flexible outer layer. The left half of the hose-like respiratory tract 12 represents the abdominal region while the right half represents the region of the rib cage. The thoracic-expansion measuring belt 4 is placed in the region of the rib cage while the abdominal-expansion measuring belt 5 is placed in the abdominal region.

In the unobstructed respiratory tract 12 shown in FIG. 2, the outer layer shows a substantially steady movement in the regions of the abdomen and of the rib cage, with cyclic outwards and inwards movements taking place in the rhythm of the respiratory movement. The abdominal measuring signal MA and the thoracic measuring signal MT are thus virtually in phase.

This situation is different in the event of an obstruction 11 in the trachea as it is shown in FIG. 3. The respiratory movement continues but goes against an obstructed opening, causing the amount of air locked in the respiratory tract 12 to be pushed back and forth between the lower and the upper end thereof, with the result that the abdominal measuring signal MA and the thoracic measuring signal MT are approximately opposite in phase. This is shown by the curves of both measuring signals MA and MT which are schematically plotted over time t in FIG. 3.

The anti-phase signal curves of the abdominal measuring signal MA and of the thoracic measuring signal MT are detected by the control and evaluation unit 3. The control and evaluation unit 3 determines and evaluates a phase difference PD that occurs between the two measuring signals MA and MT. To this end, the timing of the maximum value is determined which may initially require an amplification and an analog/digital conversion of the two in particular electric measuring signals MA and MT. This may be done by means of the usual methods involving mathematical calculation and/or interpolation. The sought phase difference PD is obtained by comparing the timing of the respective maximum values, thus enabling a respiratory disorder to be recognized.

When assessing a respiratory disorder that has been recognized from the phase difference PD, the control and evaluation unit 3 in particular also uses other detected values such as the flow measuring signal MF and/or the position measuring signal MK.

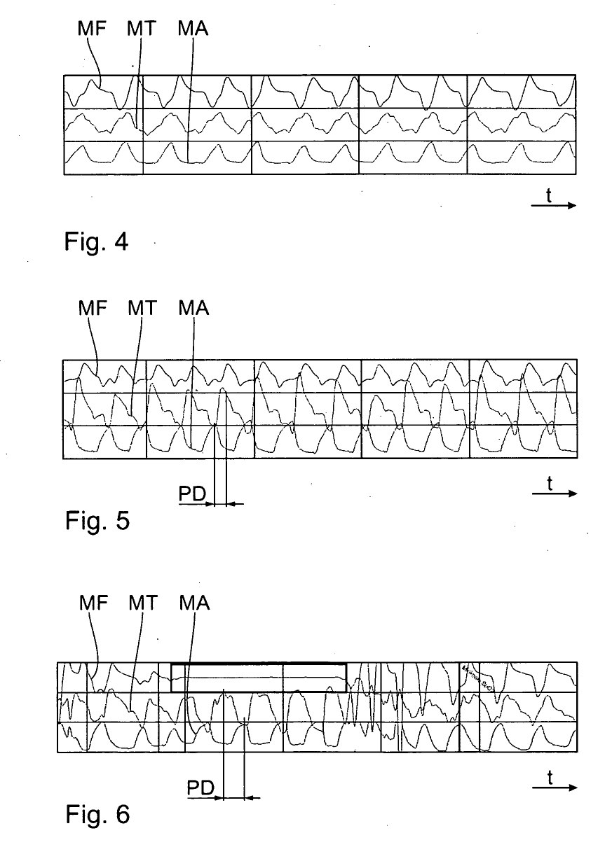

Along with the time curves of measured thoracic and abdominal measuring signals MT or MA, respectively, FIGS. 4 to 6 also show the measured time curve of the corresponding flow measuring signal MF. The conditions shown in FIG. 4 refer to unobstructed airways. The thoracic and the abdominal measuring signal MA or MT, respectively, are in phase. In this case, the phase difference PD detected by the control and evaluation unit approximately amounts to 0°.

The measuring curves according to FIG. 5 show a situation when the airways are partially obstructed. The thoracic and the abdominal measuring signal MT or MA, respectively, are staggered by a phase difference PD of approximately 120° with respect to each other. The airways are not completely obstructed, however, so that the patient is still able to breathe although this requires a considerable amount of effort. This is also shown by the measuring curve of the flow measuring signal MF. Although the flow measuring signal MF does not indicate an apnea, there is however a respiratory disorder which is recognized by the control and evaluation device 3 on the basis of the significant phase difference PD; this respiratory disorder impairs the patient's 2 sleep and is therefore also included in the respiratory report.

FIG. 6 shows the measuring curves in the event of a complete obstruction of the airways. The thoracic and the abdominal measuring signal MT or MA, respectively, are staggered by a phase difference PD of approximately 180° with respect to each other. During the interval that is marked by a frame along the curve of the flow measuring signal MF, the airways are completely obstructed, causing respiratory flow to stop. The flow measuring signal MF has a value of zero, which is indicative of an obstructive apnea. The patient 2 wakes up and starts to breathe again.

The evaluation procedure performed by the control and evaluation unit 3 on the basis of the phase difference PD enables an imminent partial or complete obstruction to be recognized at an early stage. When the phase difference PD is recorded and evaluated on a continuous basis, such an obstruction can be recognized from a change, in particular from an increase, in phase difference PD. This allows for counter measures to be taken in due time by changing, i.e. for example increasing, the ventilation pressure that is settable by means of the subunit 8 for respiratory support. This way, an imminent obstruction may even be avoided. There will be no sleep disturbance or disruption.

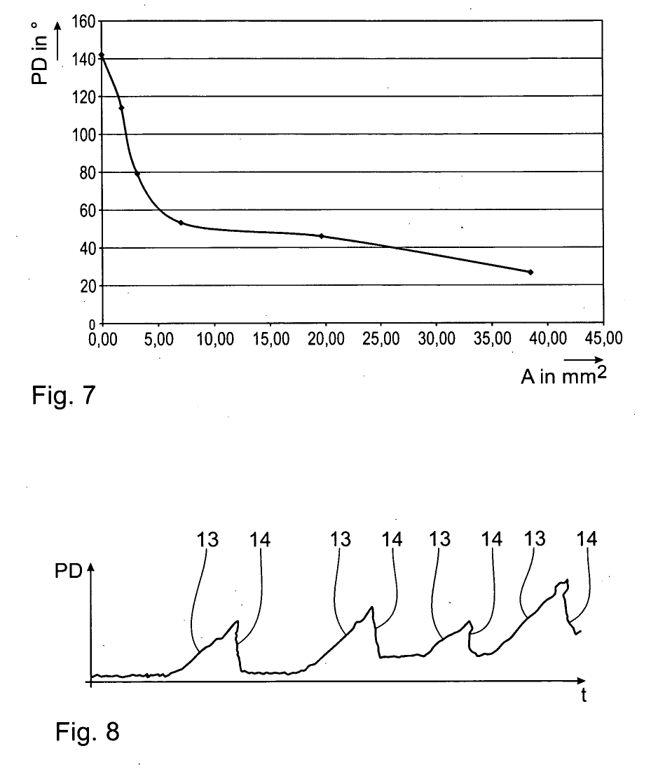

The phase difference PD not only enables an obstruction to be recognized but also allows determination of the severity of the obstruction. There is a unique monotonous relationship between the determined phase difference PD and the level of obstruction which is shown by the diagram of FIG. 7, for example. In this diagram, the determined phase difference PD is plotted over an opening surface A of an aperture with variable diameter that is introduced in the patient's 2 air supply. In the trials that were carried out by varying the aperture diameter, a simulation was performed in order to reproduce the conditions when the airways are free, partially obstructed and completely obstructed. It is obvious that in the event of phase differences PD of more than approximately 60°, the surfaces of opening A for inflow and outflow of breathing air are reduced to a very small size. The fact that contrary to expectations, the phase difference PD does not amount to approximately 180° but only to 142° at an opening surface of 0 mm², i.e. in the event of a simulated total obstruction, according to the diagram of FIG. 7, is the result of inaccuracies of the equipment used for simulating the level of obstruction. The mask and/or the aperture that are used give rise to another serial mechanical compliance which slightly falsifies the results.

A similar or the same relationship as that shown in FIG. 7 is stored in the control and evaluation unit 3 in order to determine the level of the recognized obstruction on the basis of the current recordings of phase difference PD.

Likewise, a phase threshold is stored in the control and evaluation unit 3 that may be selected from the range of between 50° to 70°. If the recorded phase difference PD exceeds this phase threshold for a period of time that is longer than a minimum threshold of for example 10 seconds but shorter than a maximum threshold of for example 120 seconds, this is regarded as indicative of an obstruction. Based on the flow measuring signal MF as well as the stored relationship between the recorded phase difference PD (described above) and the level of obstruction (described by the remaining opening surface A), the obstruction may be classified as a partial or a complete one, which is then recorded accordingly in the respiratory report. FIG. 8 shows the curve of the phase difference PD over time t. The exemplary time curve of the phase difference PD that is shown contains sections 13 of a continuously increasing phase difference PD that are each followed by a section 14 of rapidly decreasing phase difference PD. This behavior is the result of fluctuative partial or complete obstructions which cause the patient 2 to wake up so that breathing abruptly reverts back to normal.

In order to obtain timing diagrams that are suitable for evaluation, such as the diagram according to FIG. 8, provision may be made for smoothing the detected time signal of the phase difference PD by means of a moving average, for example, so as to filter out strong short-term variations which might otherwise lead to errors in measurement and diagnosis.

What is claimed is:

1. A method of monitoring a patient (2), the method comprising
a) detecting a respiratory movement of the patient (2) by means of a thoracic expansion sensor (4) that is placed near the rib cage and by means of an abdominal expansion sensor (5) that is placed near the abdomen;
b) detecting a phase difference (PD) between a thoracic measuring signal (MT) delivered by the thoracic expansion sensor (4) and an abdominal measuring signal (MA) delivered by the abdominal expansion sensor (5);
c) monitoring the detected phase difference (PD) to determine whether it fulfills at least one of the following conditions:
the phase difference (PD) exceeds at least a phase threshold;
the phase difference (PD) increases over time;
e) recognizing the presence of a disorder in the respiratory tract of the monitored patient (2) on the basis of a phase difference (PD) that fulfills at least one of the following conditions:
the phase difference (PD) exceeds the phase threshold;
the phase difference (PD) increases.

2. A method according to claim 1, wherein the phase difference (PD) is monitored continuously.

3. A method according to claim 2, wherein the phase difference (PD) is monitored to determine a variation over time thereof.

4. A method according to claim 1, wherein the phase difference (PD) is monitored continuously to detect whether a time curve of the phase difference (PD) shows a section (13) of increasing phase difference (PD) that is immediately followed by a section (14) of decreasing phase difference (PD).

5. A method according to claim 1, wherein a period of time is recorded in which the phase difference (PD) exceeds the phase threshold.

6. A method according to claim 1, wherein the severity of the disorder is determined on the basis of a value of the phase difference (PD).

7. A method according to claim 1, wherein a position of the patient's (2) body is recorded.

8. A method according to claim 1, wherein amplitudes of the thoracic measuring signal (MT) and of the abdominal measuring signal (MA) are checked to determine whether they fall below an amplitude threshold.

9. A method according to claim 1, wherein a respiratory flow of the patient (2) is recorded that is taken into account when assessing the disorder.

10. A method according to claim 1, wherein a counter measure is taken when a disorder is recognized in the respiratory tract.

11. A method according to claim 10, wherein the counter measure that is taken is an increase of a ventilation pressure.

12. An apparatus for monitoring a patient (2), comprising
a) a thoracic expansion sensor (4) to be placed near the rib cage and an abdominal expansion sensor (5) to be placed near the abdomen, each of which being used to detect a respiratory movement of the patient (2), and
b) an evaluation and control unit (3) to which the thoracic expansion sensor (4) and the abdominal expansion sensor (5) are connected, wherein
c) the evaluation and control unit (3) is designed such as to
c1) determine a phase difference (PD) between a thoracic measuring signal (MT) delivered by the thoracic expansion sensor (4) and an abdominal measuring signal (MA) delivered by the abdominal measuring sensor (5);
c2) check the determined phase difference (PD) to determine whether it fulfills at least one of the following conditions:
the phase difference (PD) exceeds at least a phase threshold;
the phase difference (PD) increases over time; and
c3) recognize the presence of a disorder in the respiratory tract of the monitored patient (2) on the basis of a phase difference (PD) that fulfills at least one of the following conditions:
the phase difference (PD) exceeds the phase threshold;
the phase difference (PD) increases.

13. An apparatus according to claim 12, wherein the evaluation and control unit (3) is designed such as to continuously monitor the phase difference (PD).

14. An apparatus according to claim 13, wherein the evaluation and control unit (3) is designed such as to monitor the phase difference (PD) to determine a variation over time thereof.

15. An apparatus according to claim 14, wherein the evaluation and control unit (3) is designed such as to monitor the phase difference to determine an increase over time thereof.

16. An apparatus according to claim 12, wherein the evaluation and control device (3) is designed such as to continuously monitor the phase difference (PD) to determine whether a time curve of the phase difference (PD) shows a section (13) of increasing phase difference (PD) that is immediately followed by a section (14) of decreasing phase difference (PD).

17. An apparatus according to claim 12, wherein the evaluation and control unit (3) is designed such as to record a period of time in which the phase difference (PD) exceeds the phase threshold.

18. An apparatus according to claim 12, wherein the evaluation and control unit (3) is designed such as to determine the severity of the disorder on the basis of a value of the phase difference (PD).

19. An apparatus according to claim 12, wherein a position sensor (7) is provided for recording a position of the patient's (2) body, the position sensor (7) being connected to the evaluation and control unit (3).

20. An apparatus according to claim 12, wherein the evaluation and control unit (3) is designed such as to monitor amplitudes of the thoracic measuring signal (MT) and of the abdominal measuring signal (MA) to determine whether they fall below an amplitude threshold.

21. An apparatus according to claim 12, wherein a flow sensor (6) is provided to detect the respiratory flow, the flow sensor (6) being connected to the evaluation and control unit (3), and wherein the evaluation and control unit (3) is designed such as to take into account the recorded respiratory flow when assessing the disorder.

22. An apparatus according to claim 12, wherein the evaluation and control unit (3) is designed such as to take a counter measure when a disorder is recognized.

23. An apparatus according to claim 22, wherein the counter measure taken by the evaluation and control unit (3) is an increase of a ventilation pressure.
